(19) United States
(12) Patent Application Publication
(10) Pub. No.: US 2009/0208575 A1
(43) Pub. Date: Aug. 20, 2009

(54) Pharmaceutical Composition Of Acid Labile Substances

(75) Inventors: Jyothi Lakshmi Gunupati, Maharashtra (IN); Suryakumar Jayanthi, Maharashtra (IN); Himadri Sen, Maharashtra (IN)

Correspondence Address: MERCHANT & GOULD PC, P.O. BOX 2903, MINNEAPOLIS, MN, 55402-0903, US

(73) Assignee: LUPIN LIMITED, Mumbai (IN)

(21) Appl. No.: 11/794,748

(22) PCT Filed: Jan. 3, 2006
(86) PCT No.: PCT/IN2006/000004

§ 371 Date: Jul. 3, 2007

(30) Foreign Application Priority Data
Jan. 3, 2005 (IN) 1208/MUM/2004

(51) Int. Cl. A61K 9/30; A61K 31/4439

(52) U.S. Cl. 424/475; 514/338

(57) ABSTRACT

A pharmaceutical composition for oral use comprising a) a core comprising an effective amount of benzimidazole and an organic stabilizing agent which is present in an amount effective to stabilize the composition, b) an intermediate layer comprising of a water insoluble polymer and an organic stabilizer, and c) an outer enteric coating layer. The organic stabilizing agent is present in the core from about 1% to about 10% by weight of the core and in the intermediate layer from about 5% to about 35% by weight of intermediate layer.

### FIELD OF THE INVENTION

The present invention relates to pharmaceutical compositions containing acid labile pharmaceutically active substances for oral use, a process for the manufacture of such compositions and a method of providing a gastric acid secretion inhibitory effect to a subject in need thereof, when using them.

### BACKGROUND OF THE INVENTION

Substituted benzimidazoles are potent inhibitors of gastric acid secretion. These compounds are susceptible to degradation and/or transformation in both acid and neutral media. The acidic decomposition of these acid labile compounds is due to an acid catalyzed reaction described by G. Rackcur et al., in Biochem. Biophys. Res. Commun. 1985: 128(1). P477-484. Therefore, such labile drugs need to be formulated in a way to stabilize the compositions.

U.S. Pat. Nos. 4,853,230 and 4,786,505 describe enteric-coated pharmaceutical formulations of acid labile substances for oral use, where the cores contain acid labile drugs mixed with alkaline reacting substances. These are coated with a first separating layer, which rapidly disintegrates in gastric fluid, and a final enteric layer.

U.S. Pat No. 5,232,706 claims an oral pharmaceutical preparation of omeprazole or an alkali salt of omeprazole and a process for producing such preparation. The design principle of the preparation is basically similar to the U.S. Pat. No. 4,786,505. This preparation is comprised of: 1) a nucleus of active drug and first basic organic compound; 2) a first coating on nucleus containing at least a layer of a basic water soluble excipient and a second basic organic compound; and 3) a second coating formed by an enteric coating. The major difference between U.S. Pat. Nos. 5,232,706 and 4,786,505 is the type of basic compound used; the former uses basic organic compound the later uses inorganic alkaline reaction compounds in core and in subcoating.

U.S. Pat. No. 5,035,899 relates to a) a core containing a pharmacologically effective amount of a pharmacologically effective, acid-unstable benzimidazole compound, b) a slightly water-soluble first coating layer, coated on the core, comprising a slightly water-soluble, film-forming material selected from the group consisting of ethyl cellulose and polyvinyl acetate and fine particles of a slightly water-soluble substance selected from the group consisting of magnesium oxide, silicic anhydride, calcium silicate, magnesium hydroxide, magnesium carbonate, aluminum hydroxide, calcium stearate, magnesium stearate and sucrose fatty acid esters suspended in the first layer, and c) a second coating layer, coated on the first layer, of an enteric polymer film.

U.S. Pat. No. 5,385,739 relates to a stable formulation of Omeprazole microgranules containing a neutral core of sugar and starch coated with an active layer of drug and mannitol powder mixture with the aid of a solution of a binding agent in water plus ethanol. An additional protective layer of mannitol and sugar syrup is then applied prior to the final gastroprotection coating.

U.S. Pat. No. 5,399,700 teaches a method for stabilizing an acid unstable benzimidazole derivative, by forming an inclusion complex of omeprazole with cyclodextrin.

U.S. Pat. No. 5,026,560 discloses spherical granules having a seed core coated with a binder and spraying powder containing lansoprazole as active drug, low substituted hydroxypropylcellulose and magnesium or calcium carbonate as alkaline agents. The powder-coated core is further coated with spraying powder of low substituted hydroxypropylcellulose and then with enteric coating agent.

U.S. Pat. No. 5,045,321 describes a pharmaceutical composition for coated tablets or granules, which is comprised of lansoprazole being in contact with at least one of the basic inorganic salts evenly. No protective and/or enteric coating is mentioned in the patent claims.

U.S. Pat. No. 5,093,132 is similar to the U.S. Pat. No. 5,045,321 but more specifically, describes an oral stabilized pharmaceutical composition for the inhibition of gastric acid secretion comprising of lansoprazole or its pharmaceutically acceptable salt in contact evenly with a basic inorganic salt stabilizing agent.

U.S. Pat. No. 5,997,903 discloses an orally administrable medicament in pellet or tablet form which is resistant to gastric juice, and in which each pellet or tablet consists of a core in which active compound or its physiologically-tolerated salt is in admixture with binder, filler and, optionally, a member selected from the group consisting of another tablet auxiliary and a basic physiologically-tolerated inorganic compound, an inert water-soluble intermediate layer surrounding the core and an outer layer which is resistant to gastric juice, wherein the active compound is pantoprazole, the binder is polyvinylpyrrolidone and/or hydroxypropyl methyl cellulose and, optionally, the filler is mannitol.

U.S. Pat. No. 6,726,927 discloses a new stable enteric coated pharmaceutical dosage forms for oral administration containing omeprazole or Lansoprazole, to a formulation essentially consisting of: a) the core formulation by dry mixing, without using an aqueous granulating solution, the acid unstable drug with the alkaline substance; b) quantitatively filling the core formulation into the hard gelatin capsule shell to give a filled hard gelatin capsules shell, wherein the gelatin capsule shell has an outer surface and an inner surface; and c) coating the outer surface of the filled hard gelatin capsule shell with the enteric coating solution or dispersion.

WO 85/03436 discloses a pharmaceutical preparation in which the core contains active drugs mixed with buffering compounds such as sodium dihydrogenphosphate, which maintains a constant pH. A coating material is used to provide a constant rate of diffusion of the pharmaceutical active. However, this formulation is not suitable for acid labile compounds where a rapid release in the small intestine is required. The direct application of an enteric coating onto the pharmaceutical active would adversely influence the storage stability of the acid labile compounds contained therein.

WO 03/077829 discloses a process for preparation of a pharmaceutical composition for oral use with desired dissolution profile and stability comprises steps of manufacturing a) core containing a pharmacologically effective acid labile compound, and/or its alkaline salts, optionally with alkaline reacting substance b) an inert sub-coating layer which is a first coating layer, coated on the core, comprising film forming material such as hydroxypropyl methyl cellulose, microcrystalline cellulose, polyvinyl pyrrolidone, hydroxymethyl cellulose, hydroxy ethyl cellulose, dextran and optionally water insoluble particles such as magnesium oxide, sililic anhydride, calcium silicate, magnesium hydroxide magnesium carbonate, aluminium hydroxide, calcium stearate and magnesium stearate c) second coat, termed as a seal coat, comprising of a mixture of polymers like hydroxypropyl methyl cellulose, cellulose acetate phthalate, and ethyl cellulose over the sub-coat d) an enteric coating layer surrounding the said seal coat layer, wherein the seal coat layer isolates the core and the sub coat layer from the enteric coating layer.

U.S. Pat. No. 6,013,281 discloses a water soluble separating layer comprising a water soluble salt of an enteric coating polymer is formed in situ between an alkaline reacting core material containing a proton pump inhibitor, such as omeprazole, lansoprazole or pantoprazole, and an enteric coating. The alkaline reacting core may be prepared in different ways, such as by preparation of granules or tablets including the active substance and the alkaline reacting compound(s) or by application of a layer including the active substance and the alkaline reacting compounds to preformed seeds.

WO 94/02140 describes an enteric pharmaceutical composition comprising a core containing an anti-ulcer agent, such as omeprazole or lansoprazole, an undercoating of one or two layers and an enteric coating, wherein the core and/or the undercoating comprises aluminium hydroxide.sodium bicarbonate coprecipitate optionally in mixture with a buffer, or a mixture of one of the following with a buffer: aluminium glycinate, an amino acid, an acid salt of an amino acid and an alkali salt of an amino acid, as a stabilizer, the buffers used being capable of controlling the pH of the mixtures to 8-9.

U.S. Pat. No. 5,626,875 describes a stable oral pharmaceutical formulation containing a benzimidazole compound, which is labile in acid medium, e.g. omeprazole and lansoprazole, which is obtained by coating inert cores with a first layer containing the benzimidazole compound, a water-soluble inert polymer such as hydroxypropyl methylcellulose or hydroxypropyl cellulose, and pharmaceutically acceptable excipients having a non-alkaline reaction, such as talc, followed by coating with a second layer comprising an inert water-soluble polymer such as hydroxypropyl methylcellulose or hydroxypropyl cellulose, talc and a pigment such as titanium dioxide, and finally with a third enteric layer comprising a polymer which is resistant to gastric juice, such as copolymerized methacrylic acid/methyl methacrylate, a plasticizer such as triethyl citrate or the like, and talc. The layers are applied using aqueous solutions or dispersions.

U.S. Pat. No. 5,385,739 discloses a dry mixture of omeprazole, mannitol, sodium lauryl sulfate and carboxy methyl starch which is applied to neutral cores of sugar and starch by means of a binder solution of hydroxypropyl methylcellulose in a mixture of water and ethanol, each application being followed by a drying step. Also protective layers of mannitol applied using the same aqueous hydroxypropyl methylcellulose binder solution as above, saccharose syrup and an enteric coating of hydroxypropyl methylcellulose phthalate are provided.

U.S. Pat. No. 6,149,942 describes an omeprazole formulation containing TiO₂ for stabilization. The stated purpose is to obtain a stable pharmaceutical formulation having a core containing omeprazole and a single coating, only. The TiO₂ is added to the omeprazole containing core and optionally also to the enteric coating. The formulation is obtained by application of an aqueous suspension containing TiO₂ and various auxiliary agents, such as binders, sedimentation retarding agents and pH correcting substances, as well as a decreasing amount of omeprazole to an initial core, so that the mixture to be applied by the end contains practically no omeprazole. After drying an enteric coating is applied.

The formulation used in the test for stability includes a substantive amount of the alkaline substance, disodium hydrogen phosphate, being known as a stabilizer to omeprazole, in addition to the TiO₂.

As illustrated by the above listing of prior art, many suggestions have been made regarding the preparation of formulations containing omeprazole and other acid labile benzimidazoles for oral administration.

It is an object of the present invention to provide yet another composition for stabilization of acid labile compound, wherein the intermediate layer is insoluble to prevent ready access of moisture to the core and in addition contains an alkaline reacting compound to provide as a buffering layer between the acidic enteric coating and the alkaline core. Another object of the present invention is a method of providing a gastric acid secretion inhibitory amount of acid-labile substance to a subject in need thereof.

### SUMMARY OF THE INVENTION

Thus according to one aspect of the invention there is provided a pharmaceutical composition for oral use comprising:
a) a core comprising an effective amount of acid-labile pharmaceutically active substance and an organic stabilizing agent which is present in an amount effective to stabilize the composition
b) an intermediate layer comprising of a water insoluble polymer and an organic stabilizer
c) an outer enteric coating layer

According to another aspect of the invention there is provided a process for the preparation of an oral pharmaceutical composition comprising blending the acid labile compound with diluents, granulating with a solution of binder and organic stabilizer, drying the granules, lubricating them and compressing into tablets, wherein these core tablets are coated with an intermediate layer comprising water insoluble polymer and an organic stabilizing agent and is further coated with an enteric polymer.

According to a further aspect of the invention there is provided a method of providing a gastric acid secretion inhibitory amount of active to a subject in need thereof, comprising: orally administering to the subject a pharmaceutical composition comprising:
a) a core comprising an effective amount of acid-labile pharmaceutically active substance and an organic stabilizing agent which is present in an amount effective to stabilize the composition
b) an intermediate layer comprising of a water insoluble polymer and an organic stabilizer
c) an outer enteric coating layer

According to a still further aspect of the invention there is provided a pharmaceutical composition for oral use comprising:
a) a core comprising an effective amount of benzimidazole or salts thereof and an organic stabilizing agent which is present in an amount effective to stabilize the composition
b) an intermediate layer comprising of a water insoluble polymer and
c) an outer enteric coating layer
   characterized in that the intermediate layer also comprises from about 5% to about 35% by weight of organic stabilizing agent.

### DETAILED DESCRIPTION OF THE INVENTION

The acid labile compounds of the present invention belong to the therapeutic category of proton pump inhibitors structurally defined as benzimidazole derivatives, including lansoprazole, pantoprazole, rabeprazole, omeprazole and esomeprazole as prototype examples. These acid unstable compounds are mixed with effective amount of organic stabilizers to stabilize the composition.

Core defined in the present invention may be a granule; micro tablet; pellet; a tablet

The organic stabilizers of the core used in the invention include meglumine, tromethamine or mixtures thereof. The effective amount of these stabilizers in the core ranges from about 1% to about 10% by weight. The core further comprises pharmaceutically acceptable diluents, disintegrants, lubricants and binders thereof.

Pharmaceutically acceptable diluents used in the present formulation are well known to a person skilled in the art. As examples of these diluents the following can be mentioned; mannitol, lactose, microcrystalline cellulose, dicalcium phosphate, starch, pregelatinized starch, sorbitol or mixtures thereof.

The disintegrants used in the core are preferably of the so-called superdisintegrant type being well known to a person skilled in the art. As examples of these disintegrants the following can be mentioned; cross-linked polyvinylpyrrolidones, modified starches, particularly sodium starch glycolate, modified celluloses and LHPC (low substituted hydroxypropyl cellulose).

Croscarmellose sodium is e.g. commercialized under the trade name Ac-Di-Sol and sodium starch glycolate under the trade names Primojel and Explotab. Kollidon CL and Polyplasdone XL are commercial crospovidone products.

Binders used in the present formulation are well known to a person skilled in the art as exemplified can be hydroxypropyl cellulose, hydroxy ethyl cellulose, ethyl cellulose, hydroxypropyl methylcellulose, methylcellulose or mixtures thereof.

Lubricants used in the present formulation are well known to a person skilled in the art as exemplified can be calcium stearate, magnesium stearate, sodium stearyl fumarate, talc, colloidal silicon dioxide or mixtures thereof.

Cores prepared in the present formulation are further coated with an intermediate layer, which comprises mixture of water insoluble polymers and organic stabilizing agent.

The water insoluble polymer may be present from about 5% to about 75% by weight of intermediate layer. The intermediate coating may be applied in the range of about 0.1% to about 10% by weight of core tablet.

The water insoluble polymer may be selected from the group comprising ethylcellulose, polyvinyl acetate, Eudragit R, Eudragit L, Eudragit RS 30D or mixtures thereof.

The organic stabilizer of the intermediate layer used may be selected from meglumine, tromethamine or mixtures thereof. These stabilizers of intermediate layer may be present in the range of about 5% to about 35% by weight of the intermediate layer. Plasticizer used may be selected from polyethylene glycol, castor oil, dibutyl sebacate, trietyl citrate, or mixtures thereof.

Talc may be added to the intermediate layer to prevent sticking of tablets. Additionally, intermediate layer may further comprise opacifiers.

The core coated with intermediate layer is further coated with enteric coating to give a stabilized peroral preparation of an acid labile compound according to the present invention. Cellulose acetate phthalate, hydroxypropyl methyl cellulose phthalate, hydroxypropyl methylcellulose acetate succinate, polyvinyl acetate phthalate, cellulose acetate trimellitate, carboxymethyl ethylcellulose, various methacrylic acid copolymers such as methacrylic acid/methylmethacrylate copolymers and shellac are non-limiting examples of materials which may be of use for such purpose.

Prefabricated solutions for enteric coating are available commercially as Acryl-eze which contains methacrylic acid copolymer type C, talc, titanium dioxide, tri ethyl citrate, sod-bicarbonate, SLS and aerosil and is marketed by Colorcon.

The invention also relates to a process for the preparation of an oral pharmaceutical formulation as stated above, said process comprising the step of mixing the acid labile active substance uniformly with pharmaceutically acceptable diluents, disintegrants, and lubricants; then granulating with a solution of binder and organic stabilizer in a required quantity of purified water; optionally, the organic stabilizer may be mixed with the drug prior to granulation. Granules are milled to the required size and dried at 45° C. till water content is less than 2% by using Karl Fischer method. Later, these granules along with lubricants and disintegrants are compressed into tablets.

Intermediate layer dispersion prepared by dissolving water insoluble polymer in a solvent additionally containing organic stabilizer, plasticizer, and lubricant is then coated on the core to the required build-up.

Further, the coating with an enteric coating may be carried out in a conventional manner. The solution obtained by dissolving enteric material in a solvent is applied onto the intermediately coated core in a conventional manner to a required build-up. One or more of these enteric-coated tablets are then filled into pharmaceutically acceptable empty capsules.

This invention also provides a method of providing a gastric acid secretion inhibitory amount of active to a subject in need thereof, comprising orally administering to the subject a pharmaceutical composition comprising a core comprising an effective amount of a benzimidazole and an organic stabilizing agent which is present in an amount effective to stabilize the composition; an intermediate layer comprising of a water insoluble polymer and a stabilizer; and an outer enteric coating layer.

The following examples illustrate pharmaceutical compositions prepared in accordance with this invention. These examples are thus not to be read as limiting the scope of the invention.

TABLE 1
Lansoprazole Delayed Release Capsules

| | Example 1 | Example 2 | Example 3 |
|---|---|---|---|
| A) Core Formula: | % w/w | | |
| Lansoprazole | 15 | 14.7 | 13.64 |
| Mannitol | 55.8 | 54.8 | 49.09 |
| Aerosil 200 | 1 | 0.98 | 0.91 |
| Sodium starch glycolate | 10 | 9.8 | 13.64 |
| Purified water | q.s. | q.s. | q.s. |
| Meglumine | 4.2 | 5.0 | 4.55 |
| Hydroxypropylmethylcellulose | 3 | 2.94 | 2.73 |
| Sodium starch glycolate | 10 | 9.8 | 13.64 |
| Calcium stearate | 1 | 1.96 | 1.82 |
| B) Intermediate layer | | | |
| Formula (with alkaliser) | % w/w | | |
| Ethylcellulose, 7 cps | 50 | 50 | 30 |
| Purified Talc | 33.7 | 33.7 | 40 |
| Meglumine | 11.3 | 11.3 | 25 |
| Tri ethyl citrate | 5 | 5 | 5 |
| Isopropyl alcohol | q.s. | q.s. | q.s. |
| Water | q.s. | q.s. | q.s. |

| C) Enteric Coating Formula | % w/w of core |
|---|---|
| Acryl-eze White | 10-15 |
| Purified water | q.s. |

Mix the sifted Lansoprazole, mannitol, aerosil, part of sodium starch glycolate in a suitable mixer to get drug mix. Dissolve meglumine and hydroxypropylmethylcellulose in sufficient quantity of water and add to the powder mix till required granules are formed. Dry these granules at 45° C. till water content (by Karl Fischer) is less than 2%. Sift the dried granules to a required size and add rest of sodium starch glycolate, calcium stearate and compress the blend using suitable punches into tablets. Intermediate coating solution is prepared by dissolving ethylcellulose in sufficient quantity of isopropyl alcohol. Add talc and triethyl citrate to the above mixture under stirring. Dissolve meglumine in sufficient quantity of purified water and add to the above mixture under stirring. Coat the tablets using coating dispersion to get a required build-up. Enteric coating is done on these tablets using a dispersion of Acryl-eze white in purified water to a required build-up.

Dissolution Study: Following the USP method for delayed release products, the formulations released more than 95% of the drug in pH 6.8 buffer within 30 minutes. The formulations were found to be storage stable under the conditions as given in the table 2.

TABLE 2
Stability Data: Stability data of Example 1

| | Initial | After 3 months 40° C./75% RH |
|---|---|---|
| Assay | 99.8 | 100 |
| Impurity A | 0.18 | 0.25 |
| Unknown | 0.03 | 0.07 |
| Total | 0.29 | 0.69 |
| % Water | 4.2 | 3.1 |

TABLE 3
Lansoprazole Delayed Release Capsules

| Name of the Ingredient | Example 4 | Example 5 |
|---|---|---|
| A) Core | % w/w | |
| Lansoprazole | 15 | 15 |
| Mannitol | 57.4 | 57.4 |
| Aersoil | 1 | 1 |
| Sodium starch glycolate (intra granular) | 10 | 10 |
| Purified water | q.s. | q.s. |
| Tromethamine | 2.6 | 2.6 |
| Hydroxypropyl methyl cellulose | 3 | 3 |
| Sodium starch glycolate (extra granular) | 10 | 10 |
| Calcium stearate | 1 | 1 |
| B) Intermediate layer (With alkaliser) | % w/w | |
| Ethyl cellulose | 60.5 | 71.43 |
| Purified Talc | — | 14.28 |
| Tromethamine | 15.2 | — |
| Polyethylene glycol | 18.2 | — |
| Diethyl pthalate | — | 14.28 |
| Sodium starch glycolate | 6.1 | — |
| Isopropyl alcohol | q.s. | q.s. |
| Purified Water | q.s. | q.s. |

| C) Enteric coating Composition | Qty. Per 500 g core |
|---|---|
| Eudragit L 30 D 55 | 187.5 g |
| Polyethylene glycol | 5.625 g |
| Talc | 14.06 g |
| Titanium dioxide | 2.81 g |
| Purified water | q.s. |

Mix the sifted Lansoprazole, mannitol, aerosil, part of sodium starch glycolate in a suitable mixer to get drug mix. Dissolve tromethamine and hydroxypropylmethylcellulose in sufficient quantity of water and add to the powder mix till required granules are formed. Dry these granules at 45° C. till water content (by Karl Fischer) is less than 2%. Sift the dried granules to a required size and add rest of sodium starch glycolate, calcium stearate and compress the blend using suitable punches into tablets. Intermediate coating solution is prepared by dissolving ethylcellulose in sufficient quantity of isopropyl alcohol. Add talc and diethyl phthalate to the above mixture under stirring. Dissolved tromethamine in sufficient quantity of purified water and add to the above mixture under stirring. Coat the tablets using coating dispersion to get a required build-up. Enteric coating is done on these tablets using a dispersion of Eudragit in purified water to a required build-up.

1. A pharmaceutical composition for oral use comprising:
a) a core comprising an effective amount of acid-labile pharmaceutically active substance and an organic stabilizing agent which is present in an amount effective to stabilize the composition
b) an intermediate layer comprising of a water insoluble polymer and an organic stabilizer
c) an outer enteric coating layer

2. A pharmaceutical composition according to claim 1, wherein the acid labile pharmaceutically active substance is a benzimidazole.

3. A pharmaceutical composition according to claim 2, wherein the benzimidazole is selected from the group comprising omeprazole, esomeprazole, lansoprazole, rabeprazole and pantoprazole or their corresponding enantiomers.

4. A pharmaceutical composition according to claim 1, wherein the organic stabilizing agent is selected from the group consisting of meglumine, tromethamine or mixtures thereof.

5. A pharmaceutical composition according to claim 1, wherein the organic stabilizing agent is present in the core from about 1% to about 10% by weight of the core and in the intermediate layer from about 5% to about 35% by weight of intermediate layer.

6. A pharmaceutical composition according to claim 1, wherein the intermediate layer comprises a water insoluble polymer selected from ethylcellulose, polyvinyl acetate, Eudragit RS, Eudragit RL or mixtures thereof.

7. A pharmaceutical composition according to claim 6, wherein water insoluble polymer is present from about 5% to about 75% by weight of intermediate layer.

8. A pharmaceutical composition according to claim 1, wherein the intermediate layer is present in the range of about 0.1% to about 10% by weight of core tablet.

9. A pharmaceutical composition according to claim 1, wherein the enteric coating comprises hydroxypropylmethylcellulose phthalate, cellulose acetate phthalate, cellulose acetate trimaleate, co-polymerized methacrylic acid/methacrylic acid methyl ester or polyvinyl acetate phthalate, optionally contains a plasticizer.

10. A pharmaceutical composition according to claim 1, wherein the core further comprises pharmaceutically acceptable diluents, disintegrants, binders, lubricants etc.

11. A pharmaceutical composition according to claim 10, wherein the diluents are selected from the group comprising mannitol, lactose, microcrystalline cellulose, dicalcium phosphate, starch, pregelatinized starch, sorbitol or mixtures thereof.

12. A pharmaceutical composition according to claim 10, wherein the disintegrants are selected from the group comprising sodium starch glycolate, croscarmellose sodium, crospovidone, starch or mixtures thereof.

13. A pharmaceutical composition according to claim 10, wherein the binders are selected from the group comprising hydroxypropyl cellulose, hydroxy ethyl cellulose, ethyl cellulose, hydroxypropyl methylcellulose, methylcellulose or mixtures thereof.

14. A pharmaceutical composition according to claim 10, wherein the lubricants are selected from the group comprising calcium stearate, magnesium stearate, sodium stearyl fumarate, talc, colloidal silicon dioxide or mixtures thereof.

15. A pharmaceutical composition according to claim 1, wherein the intermediate layer further comprises plasticizer and lubricant thereof.

16. A pharmaceutical composition according to claim 15, wherein the plasticizer is selected from the group comprising polyethylene glycol, castor oil, dibutyl sebacate, triethyl citrate or mixtures thereof.

17. A pharmaceutical composition according to claim 15, wherein the lubricant is selected from the group comprising calcium stearate, magnesium stearate, sodium stearyl fumarate, talc, colloidal silicon dioxide or mixtures thereof.

18. Process for the preparation of an oral pharmaceutical composition comprising blending the acid labile compound with diluents, granulating with a solution of binder and organic stabilizer, drying the granules, lubricating them and compressing into tablets, wherein these core tablets are coated with an intermediate layer comprising water insoluble polymer and an organic stabilizing agent and is further coated with an enteric polymer.

19. A process for preparing oral pharmaceutical composition according to claim 18, wherein one or more coated pharmaceutical composition(s) is/are filled in a pharmaceutically acceptable capsule.

20. A method of providing a gastric acid secretion inhibitory amount of active to a subject in need thereof, comprising:
orally administering to the subject a pharmaceutical composition comprising:
a) a core comprising an effective amount of acid-labile pharmaceutically active substance and an organic stabilizing agent which is present in an amount effective to stabilize the composition
b) an intermediate layer comprising of a water insoluble polymer and an organic stabilizer
c) an outer enteric coating layer

21. A pharmaceutical composition for oral use comprising:
a) a core comprising an effective amount of a benzimidazole or salts thereof and an organic stabilizing agent which is present in an amount effective to stabilize the composition
b) an intermediate layer comprising of a water insoluble polymer and
c) an outer enteric coating layer
characterized in that the intermediate layer also comprises from about 5% to about 35% by weight of organic stabilizing agent.
